(19) United States
(12) Patent Application Publication
(10) Pub. No.: US 2020/0353061 A1
(43) Pub. Date: Nov. 12, 2020

(54) NEOANTIGEN PREPARATIONS AND USES THEREOF

(71) Applicants: Mark A. Suckow, Lexington, KY (US); Ashley Kalinauskas, Vernon, CT (US); Ryan Michael Clauson, Wethersfield, CT (US)

(72) Inventors: Mark A. Suckow, Lexington, KY (US); Ashley Kalinauskas, Vernon, CT (US); Ryan Michael Clauson, Wethersfield, CT (US)

(21) Appl. No.: 16/870,726

(22) Filed: May 8, 2020

Related U.S. Application Data
(60) Provisional application No. 62/846,345, filed on May 10, 2019.

(51) Int. Cl. A61K 39/00; A61K 39/39

(52) U.S. Cl. CPC A61K 39/0011; A61K 2039/552; A61K 39/001111; A61K 39/39

(57) ABSTRACT

Improved and more robust vaccine preparations (such as cancer vaccines), and methods of treating a subject having a neoplastic cell growth, such as a cancer or tumor, are presented. A neoantigen component having one or more identified neoantigens that are overexpressed in a tumor cell or cancer cell is provided. The overexpressed neoantigens are in some examples MEW Class I neoantigens. Personalized neoantigen preparations for a subject to be used with the vaccine are also provided. Methods of screening a biological sample from a subject having a cancer or tumor to identify specific neoantigens overexpressed after treatment with the tissue vaccine are presented. A panel of neoantigens characteristic of a specific cancer or tumor is also provided. The preparations, methods and panels are provided for subjects capable of or at risk of developing a tumor, cancer, or related disease. Methods and preparations for treating malignant, pre-malignant, and at-risk subjects are also presented.

### FIELD OF THE INVENTION

The present invention claims priority to U.S. Provisional Patent Application 62/846,345 filed May 10, 2019 and relates to the field of immunotherapy agents, particularly preparations containing an autologous personalized panel of neoantigens.

### BACKGROUND OF THE INVENTION

Neoantigens, or tumor-specific antigens, are derived from random somatic mutations in tumor cells and are not present in normal cells. It has been considered that neoantigens may play a role in cancer immunotherapy. Through identifying specific neoantigens in a patient's tumor tissue, particular antigens in the tumor may be selected and incorporated into a vaccine. Where the particular selected tumor antigens are found to invoke a tumor-specific or cancer-specific immune response in the animal, the selected tumor antigens may be defined as a neoantigen, and may be used to provide a neoantigen vaccine.

Personalized neoantigen vaccines provide a tool for providing treatment to a patient during early stages of their cancer disease. These neoantigens, extremely targeted peptide sequences, have been reported to increase therapeutic efficacy and decrease potential risk of central and peripheral tolerance and adverse events associated with autoimmunity. However, the ‘real-time’ efficacy of these preparations is typically assessed in patients that have failed multiple prior chemotherapy modalities, radiation and potentially immunotherapy. This, unfortunately, results in the creation of a resistant tumor microenvironment in the patient.

The immunogenicity of neoantigens leading to T cell response has been demonstrated in humans, and a number of studies report neoantigen-specific cytotoxic T-lymphocytes (CTLs) represent the most potent tumor rejection T cell populations. However, naturally occurring neoantigen-specific CTLs in patients are typically rare, likely because of low clonal frequency and inefficient presentation of neoantigens. Therefore, a cancer vaccine, and in particular, an autologous tumor vaccine (ATV) or autologous cancer vaccine (ACV), could potentially potentiate potent immunity against neoantigens. As such, a more effective, robust cancer immunotherapy therapeutic could be provided.

A critical medical need remains for preparations/techniques that provide more robust and consistent response in an animal to cancer treatment. Identification and development of neoantigen-containing preparations would provide an improved approach for cancer treatment of a patient at earlier stages of cancer diagnosis, rather than treating the patient to conventional treatments that create a tumor resistant environment.

The medical arts remain in need of improved therapeutic preparations that incorporate customized preparations of neoantigens for patients. While personalized neoantigens are used to treat some forms of cancer, much room for improvement remains. Some of these extremely targeted peptide sequences have been described as increasing therapeutic efficacy. However, such treatments have been examined only in patients that have been exposed to multiple failed arms of chemotherapy and radiation. Such a patient, unfortunately, presents a much more tumor resistant microenvironment. Therefore, the potential for a more robust patient anti-tumor/anti-cancer response from early neoantigen therapy remains unexplored. A critical need to treat patients at earlier stages of their cancer diagnosis, as well as to devise preparations and methods that elicit a specific booster immunity effect in a subject, remains. Optimally, alternative approaches will also provide for more effective activation of the particular cell types involved in combating a subject's cancer.

### SUMMARY OF THE INVENTION

The present invention meets the above and other important medical needs in the art.

In a general and overall sense, immune therapy preparations are provided that comprise an antigenic component. This antigenic component may comprise a tissue preparation, for example, a tissue preparation of a tumor tissue. The immune therapy preparation may further comprise an augmenting agent, specifically an agent that augments the activity of the antigenic component. In some embodiments, the augmenting agent is an agent that augments the immune-promoting activities of the tissue preparation. A cancer vaccine comprising these components is also provided.

In some embodiments the augmenting agent may comprise an immune cell-specific component. By way of example, such an immune cell-specific component may comprise a preparation of neoantigen or group of neoantigens having specific antigenic specificity for a tumor antigen, the neoantigens having been identified as being overexpressed in a tumor tissue. In some embodiments, the neoantigen preparation is provided with a cancer vaccine comprising a tumor tissue preparation. The neoantigen or group of neoantigens may be further described as having high genetic identity with antigens identified to be overexpressed in a tumor tissue of a veterinary animal or human subject.

In other embodiments, the immune therapy preparation may further comprise an adjuvant. By way of example, such an adjuvant may comprise extracellular matrix (ECM) material.

A cancer vaccine comprising the immune therapy preparation is also provided. The cancer vaccine comprises a tumor tissue preparation and a neoantigen component. The neoantigen component shall comprise a neoantigen or group of neoantigens identified in a patient and/or subjects' tumor tissue that correspond to genes that are overexpressed in the tissue, relative to the expression levels of other genes in the tumor tissue.

As part of a cancer treatment regimen, a vaccine booster preparation may be provided to a subject animal and/or patient before, after or both before and after administration of the cancer vaccine. Alternatively, the cancer treatment regimen may not include the administration of a booster preparation at all. As used in the present description, a vaccine booster comprises a preparation of the neoantigen or group of neoantigens identified in a patient and/or subjects' tumor tissue that correspond to genes that are overexpressed in the tissue, relative to the expression levels of other genes in the tumor tissue. A detailed gene expression analysis of the genes in a tumor tissue specimen may be employed to identify and select the appropriate neoantigens to be included in a particular booster vaccine.

In some embodiments, the cancer vaccine and the booster vaccine preparation may be further described as comprising neoantigens that are specific for peptide fragments derived from cytosolic proteins displayed by major histocompatibility complex (MHC) Class I molecules on the surface of a cytotoxic cell. The MHC Class I molecules are found on the surface of all nucleated cells. The displayed peptide fragments will trigger an immediate response from an animal's immune system, specifically by cytotoxic T cells, against the non-self antigens being displayed. Because MHC class I molecules present peptides derived from cytosolic proteins, the pathway of MHC class I presentation is often called the cytosolic or endogenous pathway.

The cancer vaccine and neoantigen booster preparations disclosed here may comprise neoantigens that are presented on rare neoantigen-specific cytotoxic lymphocytes (CTLs). In some embodiments, the neoantigens of the cancer vaccine and the neoantigen booster preparation comprises a panel of about 5 to about 30, 40, 50, or about 100 specific neoantigens, these neoantigens being identifiable and specific for a particular tumor or tumor type in a subject animal or human patient.

In some embodiments, the antigen component is prepared by first characterizing the antigenic characteristics of a tumor identified in a subject. The subject may be any animal, including canine, bovine, equine, feline, as well as human subjects, that have been diagnosed with cancer. These cancers may include virtually any cancer, and particularly a mammary adeno carcinoma.

By way of example, non-human animal mammary adeno-carcinoma is a cancer that has nearly identical pathology, genomic mutations and metastatic potential as a carcinoma identified in human breast cancer. By way of example, the non-human mammary adeno-carcinoma may be a canine mammary adenocarcinoma. Because of the nearly identical activity, autologous canine cancer cell antigens in an autologous vaccine for a canine subject is expected to be predictive of the efficacy of an autologous human vaccine containing corresponding and specifically selected human cancer cell carcinoma antigens.

As used in the description of the present invention, a selected or specific neoantigen or group of selected or specific neoantigens relates to an antigen that is specific for a particular cancer and/or tumor (malignant or pre-malignant) in a subject, such as a veterinary or human animal subject. Autologous cancer vaccines contain an abundant variety of the subject's specific tumor (malignant or pre-malignant) or cancer tissue presented and/or associated antigens.

In one aspect, a method of prophylactically treating a subject to prevent or inhibit development of a disease is provided. By way of example, the method may be used to prophylactically treat a subject so as to prevent the development of a particular type or group of cancers. In some embodiments, the method would comprise providing a vaccine, such as a cancer vaccine, comprising a tissue preparation and a neoantigen component to a subject, the tissue preparation and the neoantigen component having antigenic characteristics specific for an identified carcinoma or malignant or pre-malignant tumor type against which the subject is to be prophylactically treated to prevent and/or inhibit. The neoantigen component will have the antigenic characteristics of one or more neoantigens that have been identified to be overexpressed in an identifiable cancer type or malignant or pre-malignant tumor type against which the subject is to be prophylactically protected against.

This method may optionally also include administering a neoantigen component without a cancer vaccine, before administration of a cancer vaccine, after administration of a cancer vaccine, or both before and after administration of a cancer vaccine. In this manner, the neoantigen component may serve to provide a booster vaccine to the subject, acting to stimulate the immune system of the treated subject. Such may be expected to result in the production of protective antibodies in the treated subject, as well as to activate immune cell types (T-cells) in the treated subject. One role of the T cell arm of the immune response is to identify and destroy neoplastic cells. T cells can also recognize peptide fragments of antigens that have been taken up by antigen presenting cells (APC), through the process of phagocytosis or pinocytosis. The way the immune system has evolved to permit T cells to recognize neoplastic cells is to require that the T cell recognize both a self-component and a microbial structure. The elegant solution to the problem of recognizing both a self-structure and a microbial determinant is the family of MHC molecules. MHC molecules (also called the human leukocyte-associated [HLA] antigens) are cell surface glycoproteins that bind peptide fragments of proteins that either have been synthesized within the cell (class I MHC molecules) or that have been incorporated or ingested by the cell and proteolytically processed (class II MHC molecules). The unique neoantigens presented by class I MHC molecules on the surface of a cell identified to be neoplastic (particularly a malignant or pre-malignant neoplasia, cancer or tumor growth and/or mass, or a non-malignant cell/cell mass), will be identified and isolated, and then provided in a preparation to be administered to a subject. In this manner, a more robust and enhanced response by the population of T cells in the subject will result. This enhanced T-cell response provides a targeted attack against the neoplastic cell or cell mass (such as a malignant and/or premalignant tumor and/or cancer, or non-malignant cell/cell mass), compared to T cell response in a subject not having received a neoantigen-containing preparation or neoantigen component. Neoplastic relates to a cell that divides more than they should or do not die when they should. Frequently, neoplastic cells form a mass of cells, referred to as a tumor. Neoplastic cells and neoplastic cell masses thereof may be benign (not cancer), precancerous, and/or malignant (cancer), and may be invasive, metastatic, non-invasive, or otherwise characterized.

The tumor and/or cancer tissue component of the cancer vaccines described herein as being an autologous tissue (for example, prepared for a tumor tissue harvested from a subject to which the preparation will be administered), a non-autologous tissue, or a combination of an autologous tissue and a non-autologous tissue. In addition, the non-autologous tissue component may be a naturally occurring tissue, such as a tissue obtained from an animal or human that will not be receiving the cancer vaccine. Alternatively, the non-autologus tissue component may be a synthetically produced tumor and/or cancer tissue. In this regard, it is envisioned that a synthetically produced tumor and/or cancer tissue may be prepared as a 3-dimensional tumor and/or cancer synthetic tissue, and provided in the presently described tissue vaccines and preparations.

In another aspect, a method of treating or inhibiting a cancer or tumor in a subject is provided. In some embodiments, the method may comprise treating the subject with a cancer vaccine according to the techniques described herein. The treatment method may include the administration of a personalized and/or customized cancer vaccine to a subject over a defined period of time according to a defined treatment regimen appropriate for the animal being treated and/or the type of tumor being treated. The treatment method may or may not also provide for the administration of a booster vaccine before, after, or both before and after, administration of the cancer vaccine. For example, a booster vaccine may be administered several days (for example, 3 to 4 days, or 4 to 14 days, 70-80 hours) or several months (2-4 months) prior to, after, or both prior to and after, administration of a cancer vaccine.

In one aspect, a booster vaccine is provided comprising one or more neoantigens identified to be the same or substantially similar to antigens that are overexpressed in a tumor tissue or tumor tissue type identified in a subject to be treated. In some aspects, the cancer vaccine may comprise an autologous tumor tissue preparation and a neoantigen component, to provide an autologous cancer vaccine (ACV). In another aspect, a non-autologous cancer vaccine (NCV) may be provided comprising a neoantigen preparation and a tumor tissue preparation prepared from tumor tissue not obtained from the subject to receive the treatment. The neoantigen preparation may comprise a neoantigen or group of neoantigens that have been found to be overexpressed in a tumor tissue of a tumor that is of a similar type or similar origin as the tumor identified in the subject to be treated.

By way of example, a previously prepared cancer vaccine comprising a tissue component (prepared from non-autologous tumor tissue) and a neoantigen component may be administered to a subject. Such preparations of cancer vaccine and neoantigen component may be prepared ahead of time and made available to the veterinarian, physician, nurse, technical staff, qualified veterinary or medical care provider, or other care giver. These preparations would therefore not have been prepared from the subject's (patients) tissue (e.g., not autologous), and used in the described methods. Alternatively, the subject may be administered an autologous cancer vaccine comprising neoantigens and a tissue component derived from a tumor tissue specimen obtained from the subject's (patients) tumor (autologous). The cancer vaccine may or may not further comprise an adjuvant. By way of example, the adjuvant may comprise extracellular matrix (ECM).

In some embodiments, the tumor tissue component of the cancer vaccine may comprise a previously prepared non-autologous tumor tissue component and a. neoantigen that comprises antigenic components identified to be shared among a number of similar tumor types obtained from a representative number of patients/subjects. It is envisioned that a neoantigen product as an off-the-shelf neoantigen preparation may be used through the present invention, the neoantigen product having a relatively similar antigenic profile as the class/type of tumor that has been identified in a particular tumor type among a group of subjects and/or patients. The cancer vaccine may include an adjuvant, such as an adjuvant comprising ECM. By way of example, an ECM is described in U.S. Pat. No. 8,062,646. This material may be used as an adjuvant in the cancer vaccines described herein. The teachings of this patent are specifically incorporated herein by reference in its entirety.

A peripheral blood mononuclear cell (PBMC) is any peripheral blood cell having a round nucleus. These cells consist of lymphocytes (T cells, B cells, NK cells) and monocytes. PBMC isolates prepared form animals before treatment with the cancer vaccine and after treatment with the cancer vaccine (Pre- and post-PBMC isolates, pre and post administration of the previously prepared (non-autologous) ECM vaccine or the autologous ECM vaccine) obtained from the subject being treated will be analyzed and specifically quantified to identify the neo-antigens that the subject has begun to mount in the immune response. In this manner, identified specific neoantigens identified in the isolate will be included in a neoantigen booster preparation that further includes an extracellular matrix (ECM) component. This anti-neoantigen booster may then be administered to a subject or patient, resulting in an increase in the subject of anti-tumor moieties in the subject, such as anti-cancer antibodies. As such, the subject's immune system has been primed or “activated”, such that upon subsequent administration of a cancer vaccine, an enhanced immune-response against the tumor in the subject will be launched. In addition, the subject may be further treated with the booster preparation after the cancer vaccine has been provided. In this manner, it is expected that the anti-tumor response in the subject will be even further enhanced.

Post-autologous vaccine treatment specimens of PBMCs obtained from a subject will be employed to identify specific neoantigens that are expressed as a result of the treatment, these unique overexpressed neoantigens will be selected. These specific unique neoantigens may be identified in a subject's sample of PBMC's and TCL's by performing a 2-D western blot analysis. A subjects PBMCs and TCLs will also be isolated and analyzed pre- and post-autologous vaccine treatment (AVT) by methods such as 2-D western blot, flow cytometry, ELISA and ELISpot. The unique neoantigens may be identified by comparing the post-treatment neoantigen profile of the subject to the pre-vaccine treatment neoantigen profile of the subject. The tumor of the subject, as well as a normal tissue, will preferentially be genetically sequenced using whole genome and RNA-seq. Peptides/proteins/other molecules that specifically target against the subject's identified specific unique compliment of neoantigens will then be provided as part of a booster vaccine to the subject.

In some aspects, the present invention provides a therapeutic method comprising administering and/or providing a tissue cancer vaccine to a subject to provide a vaccinated subject. The tissue cancer vaccine may comprise a non-autologous or an autologous tissue preparation combined with a neoantigen component, and may or may not further include an adjuvant. Optionally, and in some embodiments, the method may provide for administering a booster vaccine before, after, or both before and after, the administration of the (tissue) cancer vaccine. Thus, the use of the term booster vaccine in the present description is not confined to preparations provided to a subject after a vaccination. The booster vaccine may comprise a selected neoantigen or group of neoantigens identified to be capable of targeting specific and unique neoantigens expressed by the subject's cancer and/or tumor. In this manner, a more robust, and specific immune-response attack of the cancer and/or tumor in the subject may be provided. By providing a neoantigen booster vaccine to a subject soon after the subject is initially treated, the cancer vaccine is expected to enhance the immune response in the treated subject. In some embodiments, a neoantigen booster vaccine is provided to the subject within 2-3 days, 4-7 days, or within 2 to 3 months, before or after an initial cancer vaccine treatment. A personalized and targeted neoantigen preparation may be prepared though identification of the neoepitopes or group of neoepitopes present on a tumor or cancer cell type according to methods known to those of skill in the art and the teachings of the present disclosure. For example, a tumor tissue biopsy obtained from a subject may be harvested and used to identify the specific neoepitopes present on the tumor, and this information employed to create an appropriate neoantigen preparation for the cancer vaccine and booster vaccine. A personalized and targeted neoepitope may be created within about 90 days of receiving a tumor tissue biopsy or other tumor tissue specimen.

The targeted and personalized features of the anti-neoantigen booster component (together with an extracellular matrix component) of the therapeutic method provided to a subject, in combination with the cancer vaccine (autologous or non-autologous), provides an even further improved method for eliminating and/or treating a cancer in the subject, compared to treatment with a neoantigen preparation without an extracellular matrix (ECM).

The combination therapeutic method, having both a cancer vaccine (autologous or non-autologous) component and a neoantigen booster component (anti-neoantigen component, such as an antibody, or other molecular moiety that results in the priming of autologous dendritic or other immune cell (e.g., adoptive T cell transfer), provides for selective attack of target neoantigens while ensuring both CD4+ and CD8+ T cell activation. Such presents an improvement over prior techniques, and a critical missing component of current neoantigen and other vaccine approaches. This approach will lead to a more robust cancer treatment platform utilizing a subject's own response system to screen and select the most potent therapeutic approach for that patient.

By way of example, a subject to be treated according to the present methods will be selected and identified as having a specific type of transitional cell carcinoma. By focusing on subjects in whom a mammary adenocarcinoma has been identified, such as a non-human subject, the safety and efficacy of the autologous cancer vaccine and booster therapeutic approach may be established for any particular subject type, such as humans. Other subject types in which this approach will be useful include felines, equine, and other animal types.

In yet another aspect, a panel of selected overexpressed neoantigens identified in a mammary adenocarcinoma tissue is provided. In some embodiments, the panel may comprise about 10 to about 90 neoantigens, or from 10 to about 50 neoantigens, or from 20 to about 30 neoantigens. The panel of overexpressed neoantigens may be used to screen a subject biological sample, such as a biological sample containing a subject's blood, or more specifically, a subjects PBMCs and or TCLs. The panel may include human canine, or other animal neoantigens.

In another aspect, a biological assay for identifying unique and/or potent neoantigens that are identified as being overexpressed in a canine carcinoma tissue is provided. In some embodiments, the biological assay provides for the detection of unique neoantigens present in a subject biological sample. The unique neoantigens may include a selected panel of from 10 to 50, or 10 to 30, neoantigens that are identified to be present in a statistically significant number of specific types of mammary adenocarcinomas.

The biological assay may be used to screen and identify neoantigens in canines. In addition, the biological assay may be used to screen a sample from a human for a specific form of carcinoma having similar characteristics as those in canines. One such cancer is breast cancer. Identified neoantigens in a canine subject may then be used to provide a cancer vaccine and booster for human subjects having breast cancer. Both of these carcinomas have similar pathology, tumor heterogeneity, local invasiveness, and a large number of genes that share a similar expression profile.

### Neoantigen Preparation—Off-The-Shelf Preparation and Subject Tissue Preparation

In yet another aspect, a neoantigen preparation is provided. In some embodiments, the neoantigen preparation may comprise a compliment of neoantigens, such as a single tumor neoantigen, or a group of neoantigens, found to be overexpressed in a particular tumor type. The neoantigen preparation may further comprise a tissue vaccine. This tissue vaccine may be an autologous tissue vaccine or a non-autologous tissue vaccine. By way of example, an off-the-shelf neoantigen preparation may comprise a group of neoantigens identified to be present in a statistically significant number of a specific type of tumor. For example, the neoantigen profile of tumor tissue obtained from several canine subjects having urothelial carcinoma may be prepared and analyzed to identify those neoantigens that are most common in a statistically significant number of those tumor tissues. A preparation of these neoantigens may then be provided as an off-the-shelf neoantigen source, and may be used as a component in a cancer vaccine and/or booster vaccine, The cancer vaccine may or may not further be combined with an. adjuvant. For example, an adjuvant that may be included in the cancer vaccine may comprise extracellular matrix (ECM).

Combination Therapeutic Treatment: In yet another aspect, methods of treating and/or inhibiting a cancer or tumor are provided that employ a combination therapeutic regimen. In some embodiments, the cancer vaccine may be used with other conventional chemotherapeutic cancer modalities, such as anti-cancer biological agents, radiation (radiotherapy), surgery, or any combination of these. The veterinarian or trained technical attendant may at the time of treatment and in monitoring the particular needs of the subject and/or patient, may select which of these treatments, are best suited for the subject and/or that illicit a most beneficial response in the subject.

In some embodiments, the chemotherapeutic agents include, among others, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, and kinase inhibitors.

In some instances, the tumor vaccine is administered continuously for 1, 2, 3, 4, 5, 6, 7, 8, 9, 10, 14, 15, 28, 30 or more days. In some instances, the tumor vaccine may be administered at predetermined time intervals for 1, 2, 3, 4, 5, 6, 7, 8, 9, 10, 14, 15, 28, 30 or more days. In some instances, the tumor vaccine is administered is administered intermittently for 1, 2, 3, 4, 5, 6, 7, 8, 9, 10, 14, 15, 28, 30 or more days. In some instances, the tumor vaccine may be administered in 1 dose, 2 doses, 3 doses, 4 doses, 5 doses, 6 doses or more. In some instances, the tumor vaccine may be described as being administered at a therapeutically effective amount.

In some instances, the tumor from which a neoantigen is identified and selected is a tumor identified from veterinary or human tumors and/or cancers. The neoantigen may be identified for virtually any type of tumor or cancer according to the present methods that is of epithelial or mesenchymal origin. By way of example, a neoantigen profile may be identified for urotheal carcinoma or colorectal cancer. In some instances, the cancer is a B-cell malignancy. These examples are not intended to be limiting, but instead exemplary, as virtually any neoantigenic profile may be discerned and used in the practice of the present methods and preparations given the teachings of the present disclosure by one of ordinary skill in the therapeutic medicine and/or oncology treatment arts.

In particular embodiments, the pharmaceutical compositions of the invention comprise a cancer vaccine having an extracellular matrix (ECM) component and a neoantigen component. The neoantigens may comprise a combination of neoantigens found to be present in a statistically significant number of tumor tissues identified to be of a similar or the same type of cancer, such as urorethral carcinoma. The pharmaceutical composition and/or formulation will therefore comprise a combination of neoantigens and a pharmaceutically acceptable carrier or excipient.

In some embodiments, excipients for use with the compositions disclosed herein include maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

The cancer vaccines are made to be compatible with a particular local, regional or systemic administration or delivery route. Thus, the vaccines may include carriers, diluents, or excipients suitable for administration by particular routes. Specific non-limiting examples of routes of administration for the vaccines are parenteral, e.g., intravenous, intra-arterial, intradermal, intramuscular, subcutaneous, intranodal, intratumoral, and other delivery types suitable for the treatment method or administration protocol.

In some embodiments, solutions or suspensions used for the parenteral application of a vaccine, or of a booster preparation, include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. In some embodiments, pH is adjusted with acids or bases, such as hydrochloric acid or sodium hydroxide.

The cancer vaccines and booster preparations for injection may include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.), or phosphate buffered saline (PBS). In some embodiments, the carrier is a solvent or dispersion medium containing, for example, water, ethanol, polyol (for example, glycerol, propylene glycol, and liquid polyetheylene glycol, and the like), or suitable mixtures thereof. Fluidity is maintained, in some embodiments, for example, by the use of a coating such as lecithin, by the maintenance of the required particle size in the case of dispersion, and by the use of surfactants. Antibacterial and antifungal agents include, for example, parabens, chlorobutanol, phenol, ascorbic acid, and thimerosal. Isotonic agents, for example, sugars; polyalcohols such as mannitol or sorbitol; or sodium chloride, in some embodiments, are included in the composition. In some cases, also included may be an agent which delays absorption, in some embodiments, for example, aluminum monostearate or gelatin prolongs absorption of injectable compositions.

In some embodiments, the sterile injectable formulations of the cancer vaccine and booster preparations are prepared by incorporating the neoantigen component in the required amount in an appropriate solvent with one or a combination of the above ingredients. Generally, dispersions are prepared by incorporating the active composition into a sterile vehicle containing a basic dispersion medium and any other ingredient. In the case of sterile powders for the preparation of sterile injectable solutions, methods of preparation include, for example, vacuum drying and freeze-drying which yields a powder of the active ingredient plus any additional desired ingredient from a previously prepared solution thereof.

The following definitions are employed in the following description:

It is understood that aspect and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X”.

As used herein the term “neoantigen” or “neopeptide” are used interchangeably and refer to a peptide expressed by a diseased or stressed cell (e.g. cancer cell).

As used herein, the term “booster” refers to a preparation that is provided to a subject before, after, or before and after, a tissue cancer vaccine.

As used herein, the term “neoplastic” relates to a cell that divides more than it should or do not die when they should. Frequently, neoplastic cells form a mass of cells, referred to as a tumor. Neoplastic cells and neoplastic cell masses thereof may be benign (not cancer), precancerous, and/or malignant (cancer), and may be invasive, metastatic, non-invasive, or otherwise characterized.

As used herein, the term “immunogen” refers to a moiety, which optionally can be administered to a subject, which induces an immunological response.

The terms “recipient”, “individual”, “subject”, “host”, and “patient”, are used interchangeably herein and in some cases, refer to any mammalian subject for whom diagnosis, treatment, or therapy is desired, particularly companion animals, such as canines.

The term “about X-Y” used herein has the same meaning as “about X to about Y.”

As used herein, reference to “not” a value or parameter generally means and describes “other than” a value or parameter. For example, the method is not used to treat cancer of type X means the method is used to treat cancer of types other than X.

As used herein and in the appended claims, the singular forms “a,” “or,” and “the” include plural referents unless the context clearly dictates otherwise.

As used herein, “treatment” or “treating” is an approach for obtaining beneficial or desired results including clinical results. For purposes of this invention, beneficial or desired clinical results include, but are not limited to, one or more of the following: prophylactically protecting a subject against a disease (e.g., against developing cancer and/or a tumor), decreasing one more symptoms resulting from the disease, diminishing the extent of the disease, stabilizing the disease (e.g., preventing or delaying the worsening of the disease), preventing or delaying the spread (e.g., metastasis) of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the invention contemplate any one or more of these aspects of treatment.

The term “individual” or “subject” is used synonymously herein to describe a mammal, including humans. An individual includes, but is not limited to, human, bovine, horse, feline, canine, rodent, or primate. In some embodiments, the individual is human. In some embodiments, an individual suffers from a disease, such as cancer. In some embodiments, the individual is in need of treatment.

As used herein, by “pharmaceutically acceptable” or “pharmacologically compatible” is meant a material that is not biologically or otherwise undesirable, e.g., the material may be incorporated into a pharmaceutical composition administered to an individual without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

These and other aspects and advantages of the present invention will become apparent from the subsequent detailed description and the appended claims. It is to be understood that one, some, or all of the properties of the various embodiments described herein may be combined to form other embodiments of the present invention.

### DESCRIPTION OF THE SEVERAL VIEWS OF THE DRAWING

FIG. 1—Flow chart demonstrating therapeutic regimen, employing an initial autologous tumor vaccine (ATV), followed by a personalized anti-neoantigen booster treatment.

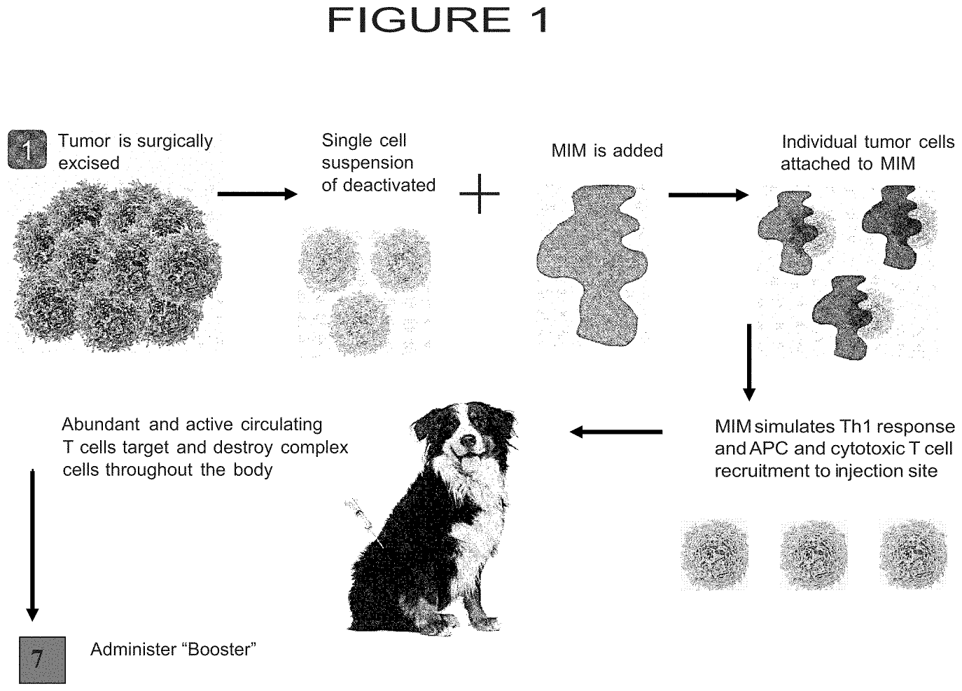

The anti-neoantigen components of the booster preparation target the identified overexpressed MEW Class I neoantigens present in a subject sample post ATV treatment.

### DETAILED DESCRIPTION OF THE INVENTION

The following examples present a description of various specific aspects of the intended invention, and are not presented to limit the intended invention in any way.

### EXANOKE 1

### Neoantigen Characterization

The present example demonstrates the utility of the present invention for priming the immune system with a cancer vaccine (autologous or non-autologous (ACV), and determining the identity of overexpressed neoantigens in a subject sample. The immune response against these identified overexpressed neoantigens may be further ‘boosted’ to enhance the therapeutic efficacy and specificity of the autologous cancer immunotherapy in a subject having a known cancer.

The cancer vaccine (autologous (ACV) or non-autologous) and neoantigen booster approach will provide a rapid alternative to conventional neoantigen screening. The addition of a booster component as part of an immune-therapy or cancer treatment regimen provides for the highest likelihood for T cell proliferation in the subject. Additionally, the present approach will permit a demonstration of the efficacy in canines that will serve to predict response and use in human tumor treatment.

Recruitment of Canine Patients

Canine patients diagnosed with transitional cell carcinoma of the bladder will be identified. Tissue samples from these animals will be obtained. A cancer vaccine (autologous (ACV) or non-autologous) preparation from each specific animal will then be prepared, and the animal will then be treated with the cancer vaccine (non-autologous or autologous cancer vaccine (ACV)). A tumor lysate will be collected, as well as pre-/post-PBMC blood isolates of the subject for further monitoring and treatment response assessment.

Functional assays of tumor cell killing (CTL and ADCC) as well as indices of immune activation and T cell proliferation (lymphocyte re-stimulation/proliferation, intracellular cytokine staining and studies to assess Th1, Th2, Th17 and T reg populations and immunophenotyping via FACS) will be conducted.

2-D Western Blots will be conducted on pre-/post-serum to isolate specific immunogenic proteins.

### EXAMPLE 2

### Full Genome Sequencing and RNA-Sequencing

Tumors from each subject will be evaluated for point, frameshift and Neo-ORF mutations. Samples will be characterized via the abundance of mutational load and predicted MHC binding peptides. This will be compared against the immunogenic protein isolates found from the 2-D Western Blot to evaluate congruencies.

Genome sequencing will be conducted on a subject's tissue using those techniques known to those of skill in the art. By way of example, these methods are described in the following references, which are specifically incorporated herein by reference: Decker et al., (2019), J.Genome Research, 25: 1646-1655; Maeda et al., (2018), BMC Cancer, 18: 472-482; Barth et al., (2016), PLoS ONE, 11 (11): e0167017; Brown and Hold, (2019), OncoImmunology, 8 (3): e1556080-e1556080-11; Dreger et al. (2016), Disease Models & Mechanisms, 9: 1445-1460).

### EXAMPLE 3

### Personalized Vaccine Peptide Boosters and Delivery Vehicle

The following parameters associated with the development of the anti-neoantigen-based booster vaccines (ACV) will be assessed:
(1) quantify pre/post blood sample changes via FACS;
(2) run functional assays on patient samples;
(3) run full genome and RNA-sequence on patient samples;
(4) compare RNA-sequence with mutated proteins isolated from 2-D western blot; and
(5) create personalized peptide boosters (months 3-6).

The creation of the personalized anti-neoantigen booster vaccines according to the present invention, are designed to augment the efficacy of autologous tumor vaccine (ATV) approaches.

### EXAMPLE 4

### Canine Personalized Neoantigen Vaccines and Booster Preparations

The present example details the steps by which the ACV will be prepared for a population of canines diagnosed with transitional cell carcinoma.

Canines diagnosed with transitional cell carcinoma, a cancer that has nearly identical pathology, genomic mutations and metastatic potential as humans, will be obtained and utilized to analyze and develop autologous or non-autologous cancer vaccine, which will include neoantigen. The treatment regimen may further include administration of a booster vaccine comprising the neoantigen. The booster vaccine may be administered before, after, or both before and after, the administration of the cancer vaccine. These preparations and treatment methods may be developed and provided for treatment of both canines and humans, or any other animal of interest having a cancer or tumor to be treated.

The present approach will ensure that treatment is provided back quickly, is targeted and personalized to the patient's specific mutational burden and by utilizing the ACV, peripheral blood mononuclear cells (PBMCs) may be collected and a comparison done to compare pre-/post-treatment enhancements to more selectively target potential neoantigens while ensuring both CD4+ and CD8+ T cell activation. This has previously been a critical missing component of current neoantigen vaccine approaches. This approach will lead to a more robust treatment platform utilizing canines to screen potential therapeutic approaches prior to treating humans.

Neoantigen Booster:

Miming the immune system with an ACV; Determination of Neoantigens or other immunoactive moiety that can be further ‘boosted’ to enhance the therapeutic efficacy and specificity of an autologous cancer immunotherapy. The cancer vaccine (non-autologous cancer composite antigen or autologous cancer vaccine (ACV)) booster approach will provide a rapid alternative to screening a specific subject tissue sample to identify overexpressed neoantigens and determining those neoantigens that have the highest likelihood for T cell proliferation. Additionally, the present approach will permit the showing of efficacy in canines prior to pursuing Phase I clinical trials in humans.

Canine patients diagnosed with transitional cell carcinoma of the bladder will be treated with an autologous cancer vaccine (ACV). A tumor cell lysate and pre-/post-PBMC blood isolates will then be collected. Canine tumors collected from veterinary clinics will be harvested and immediately shipped on ice/cold. for formulation to create a cancer vaccine, specifically, to create an autologous cancer vaccine (ACV).

Functional assays of tumor cell killing (CTL and ADCC) as well as indices of immune activation and T cell proliferation (lymphocyte re-stimulation/proliferation, intracellular cytokine staining and Luminex studies to assess Th1, Th2, Th17 and T reg populations and immunophenotyping via FACS) will be conducted on the collected samples. 2-D Western Blots will be conducted on pre-/post-serum to isolate out specific immunogenic proteins.

Full genome sequencing and RNA-sequencing. Tumors will be evaluated for point, frameshift and Neo-ORF mutations employing those techniques known to those of skill in the art. Samples will be characterized via the abundance of mutational load and predicted MHC binding peptides. This will be compared against the immunogenic protein isolates found from the 2-D Western Blot to evaluate congruencies.

Formulation of personalized peptide boosters and delivery vehicle. A personalized anti-neoantigen booster treatment for each canine will be formulated as described above.

### EXAMPLE 5

### Prophetic Human Autologous AV Booster Preparations

The present example provides a description of how the human booster ACV neoantigen preparations will be prepared according to the present invention.

The following table presents human neoantigen identified for a variety of human cancers.

TABLE 1
Human neoantigens discovered by classical approaches.

| | | Mutated | | Source | |
|---|---|---|---|---|---|
| Cancer | | gene | | of T | |
| type | Year | name | Approach | cells | Reference |
| Mela- | 1995 | CDK4 | cDNA | PBL | Wolfel |
| noma | | | library | | |
| Mela- | 1995 | MUM1 | cDNA | PBL | Coulie |
| noma | | | library | | |
| Mela- | 1996 | CTNNB1 | cDNA | TIL | Robbins |
| noma | | | library | | |
| Mela- | 1999 | CDC27 | cDNA | TIL | Wang |
| noma | | | library | | |
| Mela- | 1999 | TRAPPC1 | cDNA | PBL | Chiari |
| noma | | | library | | |
| Mela- | 1999 | TPI | Chroma- | TIL | Pieper |
| noma | | | tographic | | |
| | | | purifi- | | |
| | | | cation | | |
| Mela- | 2000 | ASCC3 | cDNA | PBL | Baurain |
| noma | | | library | | |
| Mela- | 2001 | HHAT | cDNA | TIL | Kawakami |
| noma | | | library | | |
| Mela- | 2002 | FN1 | cDNA | TIL | Wang |
| noma | | | library | | |
| Mela- | 2002 | OS-9 | cDNA | PBL | vigneron |
| noma | | | library | | |
| Mela- | 2003 | PTPRK | cDNA | TIL | novellino] |
| noma | | | library | | |
| Mela- | 2004 | CDKN2A**, | cDNA | TIL | huang |
| noma | | HLA-A11 | library | | |
| Mela- | 2005 | GAS7, | cDNA | TIL | Zhou |
| noma | | GAPDH | library | | |
| Mela- | 2005 | SIRT2, | cDNA | PBL | lennerz |
| noma | | GPNMB, | library | | |
| | | SNRP116, | | | |
| | | RBAF600, | | | |
| | | SNRPD1 | | | |
| Mela- | 2005 | Prdx5 | cDNA | PBL | sensi |
| noma | | | library | | |
| Mela- | 2011 | CLPP | cDNA | TIL | corbiere |
| noma | | | library | | [69] |
| Mela- | 2013 | PPP1R3B | cDNA | TIL | lu |
| noma | | | library | | |
| Lung | 1998 | EF2 | Chroma- | PBL | hogan |
| cancer | | | tographic | | |
| | | | purifi- | | |
| | | | cation | | |
| Lung | 2001 | ACTN4 | cDNA | TIL | echchakir |
| cancer | | | library | | |
| Lung | 2001 | ME1 | cDNA | PBL | karanikas |
| cancer | | | library | | |
| Lung | 2006 | NF-YC | cDNA | draining | takenoyama |
| cancer | | | library | lymph | |
| | | | | node | |
| Renal | 1996 | HLA-A2 | cDNA | PBL | [brandle |
| cancer | | | library | | |
| Renal | 1999 | HSP70-2 | cDNA | TIL | [Gaudin |
| cancer | | | library | | |
| Renal | 2005 | KIAA1440 | cDNA | PBL | [zhou |
| cancer | | | library | | |
| HNSCC | 1997 | CASP8 | cDNA | PBL | mandruzzato |
| | | | library | | |

TIL: tumor infiltrating lymphocytes;
PBL: peripheral blood lymphocytes;
HNSCC: Head and neck squamous cell carcinoma
**Frame-shift mutation

The above described human neoantigens will be examined for use in the cancer vaccines and booster vaccine preparations described herein for treatment of human cancers. These and other human neoantigens will be examined for the preparation and selection of a neoantigen panel that may be used to screen subject human biological samples.

The personalized anti-neooantigen vaccines and booster preparations will comprise one or more of these neoantigens, or other neoantigen as may be identified as unique to the particular human patient, together with an extracellular matrix adjuvant. The extracellular matrix adjuvant will be provided together with the neoantigen or selected neoantigen combination, as part of the vaccine and booster vaccine for treatment of a subject human having been identified to have a specific cancer.

The cancer vaccines and neoantigen preparations prepared according to the present disclosure may further comprise a pharmaceutically acceptable carrier. A “pharmaceutically acceptable carrier” denotes all substrates, solvents, diluents, excipients, adjuvants, dispersion media, and equivalent, compatible with pharmaceutical administration.

The composition to be used for human application is to be buffered so as to be suitable for human use at a physiological or slightly alkaline pH. The composition may be administered to the patient by a variety of methods, such as by subcutaneous, intradermal, intramuscular, intravenous, intraperitoneal, intratumoral, intravascular, or intra-arterial routes.

Where the vaccine is provided as an injection, the injections may be made with conventional syringes and needles, but preferentially via a Bioject.RTM. type needleless injection device.

The administration of the composition according to the invention may take place as a single or repeated dose after a certain time interval ranging from one day to one year.

The suitable dosage of the anti-neoantigen booster or other “booster” having specific immunogenically active antigens, of the cancer vaccine, such as the autologous tumor vaccine (ATV) may be adapted according to various parameters, in particular the method of administration, the composition used, the age, health, and weight of the host body, the nature and extent of the symptoms, the associated treatment type, the treatment frequency, etc., as may be indicated by the patient.

Those skilled in the art are capable of determining the suitable quantities and frequencies of administration to be provided to a subject as part of the implementation of the present compositions and techniques disclosed.

Preferentially, the composition according to the invention may be used in conjunction with radiotherapy, chemotherapy, surgery and/or further immunotherapy products such as anti-PD1 and anti-PDL1 antibodies, or the combination of a plurality of these treatments at once.

### BIBLIOGRAPHY

The following references are incorporated herein in their entirety.
1. Yong-Chen, et al (2016), Seminars in Immunology, 28: 22-27.
2. USPUB 20170321285
3. USPUB 20170224796
4. USPUB 20170202939
5. USPUB 20170224799
6. Yong-Chen Lu, Paul F. Robbins, 2016, Seminars in Immunology, 28: 22-27.
7. T. Wolfel, M. Hauer, J. Schneider, M. Serrano, C. Wolfel, E. Klehmann-Hieb, et al., Science 269 (1995) 1281-1284.
8. P. F. Robbins, M. El-Gamil, Y. F. Li, Y. Kawakami, D. Loftus, E. Appella, et al., J. Exp. Med, 183 (1996) 1185-1192.
9. S. Mandruzzato, F. Brasseur, G. Andry, T. Boon, P. van der Bruggen, J. Exp. Med. 186 (1997) 785-793.
10. D. Brandle, F. Brasseur, P. Weynants, T. Boon, B. Van den Eynde, J. Exp. Med. 183 (1996) 2501-2508.
11. J. Huang, M. El-Gamil, M. E. Dudley, Y. F. Li, S. A. Rosenberg, P. F. Robbins, J. Immunol. 172 (2004) 6057-6064.
12. K. T. Hogan, D. P. Eisinger, S. B. Cupp 3rd, K. J. Lekstrom, D. D. Deacon, J. Shabanowitz, et al., Cancer Res. 58 (1998), 5144-5150.
13. Y. C. Lu, X. Yao, Y. F. Li, M, El-Gamil, M. E, Dudley, J. C. Yang, et al., J. Immunol. 190 (2013), 6034-6042.
14. S. L. Topalian, F. S. Hodi, J. R, Brahmer, S. N. Gettinger, D. C. Smith, D. F. McDermott, et al., N. Engl. J. Med. 366 (2012) 2443-2454.
15. P. G. Coulie, F. Lehmann, B. Lethe, J. Herman, C. Lurquin, M. Andrawiss, et al., Proc. Natl. Acad. Sci. U.S.A. 92 (1995) 7976-7980.
16. R. F. Wang, X. Wang, A. C. Atwood, S. L. Topalian, S. A. Rosenberg, Science 284 (1999) 1351-1354.
17. R. Pieper, R. E. Christian, M. I. Gonzales, M. I. Nishimura, G. Gupta, R. E. Settlage, et al., J. Exp. Med. 189 (1999) 757-766.
18. J. F. Baurain, D. Colau, N. van Baren, C. Landry, V. Martelange, M. Vikkula, et al., J. Immunol. 164 (2000) 6057-6066.
19. Y. Kawakami, X. Wang, T. Shofuda, H. Sumimoto, J. Tupesis, E. Fitzgerald, et al., J. Immunol. 166 (2001) 2871-2877.
20. H. Y. Wang, J. Zhou, K. Zhu, A. I. Riker, F. M. Marincola, R. F. Wang, J. Exp. Med, 195 (2002) 1397-1406.
21. N. Vigneron, A. Ooms, S. Morel, G. Degiovanni, B.J. Van Den Eynde, Cancer Immunity 2 (2002) 9.
22. L. Novellino, N. Renkvist, F. Rini, A. Mazzocchi, L. Rivoitini, A. Greco, et al., Immunol. 170 (2003) 6363-6370.
23. J. Zhou, M. E. Dudley, S. A. Rosenberg, P. F. Robbins, J. Immunother. 28 (2005) 53-62.
24. V. Lennerz, M. Fatho, C. Gentilini, R. A. Frye, A. Lifke, D. Ferel, et al., Proc. Natl. Acad. Sci. U.S.A. 102 (2005) 16013-16018.
25. M. Sensi, G. Nicolini, M. Zanon, C. Colombo, A. Molla, I. Bersani, et al., Cancer Res. 65 (2005) 632-640.
26. V. Corbiere, J. Chapiro, V. Stroobant, W. Ma, C. Lurquin, B. Lethe, et al., (2011) Cancer Res. 71: 1253-1262.
27. H. Echchakir, F. Mami-Chouaib, I. Vergnon, J. F. Baurain, V. Karanikas, S. Chouaib, et al., (2001), Cancer Res. 61: 4078-4083.
28. V. Karanikas, D. Colau, J.F. Baurain, R. Chiari, J. Thonnard, I. Gutierrez-Roelens, et al., Cancer Res. 61 (2001) 3718-3724.
29. M. Takenoyama, J. F. Baurain, M. Yasuda, T. So, M. Sugaya, T. Hanagiri, et al., Int. J. Cancer 118 (2006) 1992-1997.
30. C. Gaudin, F. Kremer, E. Angevin, V. Scott, F. Triebel, (1999), J. Immunol. 162: 1730-1738.
31. X. Zhou, D. Y. Jun, A. M. Thomas, X. Huang, L. Q. Huang, J. Mautner, et al., (2005), Cancer Res. 65: 1079-1088.
32. USPUB 20180296653
33. USPUB 20180078624
34. USPUB 20170321285
35. USPUB 20170224796
36. USPUB 20170202939
37. USPUB 20130302376
38. USPUB 20110250229
39. USPUB 20100233203
40. USPUB 20090068682
41. USPUB 20190071502
42. Decker et al., (2019), J. Genome Research, 25: 1646-1655.
42. Maeda et al., (2018), BMC Cancer, 18: 472-482.
43. Barth et al., (2016), PLoS ONE, 11 (11): e0167017.
44. Brown and Hold, (2019), OncoImmunology, 8 (3): e1556080-e1556080-11.
45. Dreger et al. (2016), Disease Models & Mechanisms, 9: 1445-1460.

What is claimed is:

1. An immunological preparation comprising a neoantigen component and a tumor tissue component, wherein the neoantigen component comprises one or more neoantigens specific for a target neoantigen overexpressed by a tumor cell or cancer cell.

2. The immunological preparation of claim 1 wherein the target neoantigen is a neoantigen presented by an MEW Class I molecule on a tumor cell or cancer cell.

3. The immunological preparation of claim 1 further defined as a cancer vaccine.

4. A booster preparation comprising one or more overexpressed neoantigens associated with a tumor cell or cancer cell.

5. The booster preparation of claim 4 wherein the tumor cell or cancer cell is a malignant or pre-malignant tumor cell or cancer cell.

6. The booster preparation of claim 4 wherein the tumor cell or cancer cell is a mammalian tumor cell or cancer cell.

7. The immunological preparation of claim 1 comprising an adjuvant.

8. A neoantigen screening panel for a selected cancer comprising overexpressed neoantigens associated with a tumor tissue obtained from a subject having the selected cancer.

9. The neoantigen screening panel of claim 8 wherein the overexpressed neoantigens are MEW Class I neoantigens.

10. A neoantigen booster vaccine comprising one or more peptides or proteins that target neoantigens selected with the neoantigen screening panel of claim 9.

11. A method of enhancing specificity of immune response in a subject having or at risk of developing an identified carcinoma or tumor comprising:
providing a vaccine comprising a tissue preparation and a neoantigen component to the subject to provide a vaccine treated subject;
identifying overexpressed neoantigen in a biological sample obtained from the vaccine treated subject and preparing a neoantigen preparation comprising the identified overexpressed neoantigen; and
providing the neoantigen preparation to the subject,
wherein the subject provided with the neoantigen preparation has an enhanced immune response to the vaccine compared to immune response of a subject not provided the neoantigen preparation.

12. The method of claim 11 wherein the subject is a mammal.

13. The method of claim 11 wherein the tissue preparation comprises an autologous tissue preparation, a non-autologous tissue preparation, or a combination of an autologous tissue preparation and non-autologous tissue preparation.

14. The method of claim 13 wherein the non-autologous tissue preparation is a synthetic non-autologous tissue preparation.

15. The method of claim 11 wherein the cancer vaccine comprises an adjuvant.

16. The method of claim 11 wherein the neoantigen preparation is provided to the subject after administering the vaccine.

17. A method of enhancing specificity of immune response in a subject having or at risk of developing an identified carcinoma or tumor comprising:
preparing a neoantigen preparation, said neoantigen preparation comprising a neoantigen or group of neoantigens identified in a cancer cell or tumor cell similar to the identified carcinoma or tumor, wherein said neoantigen is overexpressed by the cancer cell or tumor cell;
providing the subject with the neoantigen preparation before, after, or before and after administering a vaccine to the subject, wherein the vaccine comprises a tissue component comprising an autologous or non-autologous tissue preparation of the carcinoma or tumor,
wherein the subject provided with the neoantigen preparation has an enhanced immune response to the vaccine compared to immune response to the vaccine in a subject not provided the neoantigen preparation.

18. The method of claim 17 wherein the non-autologous tissue preparation is a synthetic non-autologous tissue preparation.

19. The method of claim 17 wherein the vaccine is a cancer vaccine.

20. A neoantigen product comprising identified neoantigens overexpressed by a tumor of mesenchymal or epithelial origin.
